(19) United States
(12) Patent Application Publication
(10) Pub. No.: US 2026/0240438 A1
(43) Pub. Date: Aug. 20, 2026

(54) A WEARABLE FINGER RING DEVICE WITH MULTIPLE BIOMARKERS AND METHOD THEREOF

(71) Applicant: Sensio Enterprises Pvt Ltd, Bangalore (IN)

(72) Inventors: Kenneth JOEL, Bangalore (IN); Mohan PRABHAKAR, Bangalore (IN); Venkatesh VADDE, Bangalore (IN)

(21) Appl. No.: 19/145,120

(22) PCT Filed: Jul. 14, 2023
(86) PCT No.: PCT/IB2023/057227

§ 371 Date: Jul. 1, 2025

(30) Foreign Application Priority Data
Jul. 14, 2022 (IN) 202241040383

(51) Int. Cl. A61B 5/0205; A61B 5/00; A61B 5/021; A61B 5/024; A61B 5/0531; A61B 5/332

(52) U.S. Cl. CPC A61B 5/02055; A61B 5/0006; A61B 5/0008; A61B 5/6802; A61B 5/02108; A61B 5/02416; A61B 5/02438; A61B 5/0531; A61B 5/332; A61B 2560/0468; A61B 2562/06

(57) ABSTRACT

The present invention discloses a wearable finger ring device (100) having an optical sensing unit (101) and electrode module (102) with more than one inner electrodes (102a) and an outer electrode (102b). The optical sensing unit (101) includes light emitting diodes (LED) that illuminates the capillaries underneath the skin to collect Photoplethysmography (PPG) signal and the outer electrode (102b) of the electrode module (102) facilitates the measurement of Electrocardiogram (ECG) signal. The device (100) enables synchronous measurement of Electrocardiogram (ECG) signal and Photoplethysmography (PPG) signal, that enables estimation of Pulse Transit Time (PTT) which in combination with Pulse Wave Analysis (PWA) enables cuffless blood pressure (BP) measurement. The device (100) measures multiple vitals including heart rate, oxygen saturation (SpO₂), blood pressure, skin temperature, respiratory rate.

### DESCRIPTION OF THE INVENTION

### Technical Field of the Invention

The present invention relates generally to the wearable measurement device. More particularly, the present invention relates to a wearable finger ring device having multiple sensors to measure the vitals of human body, for estimation of the pulse transit time leading to cuffless blood pressure measurement.

### BACKGROUND OF THE INVENTION

The use of smartwatches and wristbands to track vitals throughout the day for health monitoring has changed the way of living in the past decade. These smart devices are used to track various vitals like heart rate, sleep patterns, fitness tracking etc., which has provided insight into health and fitness of the individuals through their tracking capabilities. Wearable health trackers use optical and inertial sensors embedded into a wrist worn device to track the vitals. Even though these devices provide insight to health and fitness, they do have several limitations including limited durability, comfort, low battery life, can be expensive and as they are specifically worn on the wrist, it is easier for others to see the smartwatch screen, thus invading user privacy.

In order to overcome the drawbacks of the wearable wrist tracking devices, the smart rings are developed over the decade, for measuring the vitals and tracking the activity and several other parameters of the user and enabling the user to maintain health and fitness.

The Patent Application No. U.S. Pat. No. 10,682,095 entitled “Finger ring electrocardiogram monitor trigger systems and associated methods” discloses a method of monitoring a health status of a patient using a monitoring system comprising a pair of finger ring electrocardiogram (ECG) monitors. Each finger ring monitor comprises an inner ring member and an outer ring member positioned radially outward from and operably connected to the inner ring member. The inner ring member of each monitor features a conductor characterized by at least one physiological-type sensor, and an annular bladder that defines an adjustable aperture sized to receive a left-hand finger of a patient and a right-hand finger of the patient, respectively. Upon triggering by positioning the pair of finger ring ECG monitors substantially together, the conductors of each of the finger ring ECG monitors are configured to receive biopotential signals from skin on the fingers of the patient.

The patent application no. U.S. Pat No. 6,413,223 entitled “Cuffless continuous blood pressure monitor” discloses a device for noninvasive, continuous monitoring of arterial blood pressure for advanced cardiovascular diagnoses. Most of the current noninvasive, continuous blood pressure measurement devices are mechanically intrusive or uncomfortable and, therefore, cannot be used for long-term ambulatory monitoring. This new approach requires only simple, noninvasive monitoring devices such as finger photoplethysmographs and an electrical impedance plethysmograph (EIP) to monitor the dynamic behavior of the arterial blood flow, In this approach, measured signals from these noninvasive sensors on an arterial segment are integrated to estimate the blood pressure in the segment based on a hemodynamic model. A mathematical model of the arterial blood flow is derived and transformed into a state-space representation. In the modeling, a precise hemodynamic model for the arterial segment on which sensors are located is derived, and combined with relatively simplified models of the upstream and the downstream arterial flows to represent an entire arterial stream. Then, a Kalman filter is designed based on the model and it is shown that the internal variables such as the arterial blood pressure in the arterial segment can be estimated based on the measurements, even though the observability condition of the system may not be met. Simulation results indicate that the approach can generate an accurate estimation of the arterial blood pressure in real-time even from noisy sensor signals.

The patent application no. U.S. 20180020977A1 entitled “Electrocardiogram sensor ring” discloses an electrocardiogram (ECG) sensor having a flexible thin ring-shaped substrate configured to be worn about a patient's finger. The substrate has an inner surface and an outer surface opposite the inner surface. A first ECG electrode is positioned at the outer surface of said substrate and a second ECG electrode is positioned at the inner (or outer) surface of the said substrate in contact with the finger (or the adjacent finger if the electrode is on the outer surface), whereby the two ECO electrodes receives a single-lead ECG signal when the first electrode is touched to the patient's body. Touching different locations on the body or wearing multiple rings can provide multi-lead ECG measurements. The ring shape can be converted to a wearable patch for continuous ECG measurements.

There are numerous drawbacks in the prior arts and existing systems including, incapability of synchronously recording the Electrocardiogram (ECG) and Photoplethysmography (PPG) signals. The existing systems include an optical heart rate measurement sensor (PPG) where only a few of them are capable of measuring a single lead electrocardiogram (ECG).

Therefore, there is a need for a smart wearable ring device for measuring multiple vitals of the user through multiple sensors, along with synchronous ECG-PPG signal measurement that can be used to estimate the Pulse Transit Time (PTT), where PTT is a new and useful marker capable of providing further insights into the user's health and useful in measurement of cuffless blood pressure.

### SUMMARY OF THE INVENTION

The present invention discloses a wearable finger ring device (hereafter referred to as “device”) for measuring multiple vitals of the user, the device comprises multiple sensors including but not limited to Photoplethysmography (PPG), Electrocardiogram (ECG), Bioimpedance (BioZ) and temperature sensors, capable of measuring multiple vitals including but not limited to heart rate, SpO₂, blood pressure, skin temperature, respiratory rate and so on.

The wearable finger ring device comprises an optical sensing unit having at least one multi-wavelength light emitting diode (LED) and a photodiode (PD) located at the palm side of the finger; an electrode module for biopotential and bioimpedance measurements, having at least one inner electrode on the same or opposite sides and an outer electrode; a battery and power management module to provide power supply to the device, a multi-sensor analog front end coupled with a sensor hub and primary microcontroller (MCU) and wireless transmitter.

According to the present invention, the device contains at least one light emitting diode facing the skin of the finger, that illuminates the capillaries underneath to facilitate collection the Photoplethysmography (PPG) signal. The photodiode (PD) faces the skin side of the finger that senses and collects the subtle changes in the reflected light in response to the blood flow. The device contains at least one biopotential/bioimpedance electrode located on the inner side of the wearable finger ring device in contact with the skin to facilitate sensing the signal for Bioimpedance (BioZ). The device contains an additional electrode located on the external side of the wearable finger ring device to establish contact anywhere on the body in order to facilitate the collection of Electrocardiogram (ECG) signal. The device further contains an Analog-front end (AFE) to process the collected information and a Microcontroller unit (MCU) to collect the processed information from the Analog-Front End (AFE) and transfer it to the receiving unit, for example: any electronic device, through wireless network infrastructure such as Bluetooth, General Packet Radio Service (GPRS), Wi-Fi and other systems which fall within the purview of wireless network infrastructure.

There are several advantages of the present invention including continuous measurement of multiple vitals from a single, minimally intrusive measurement site. Another advantage is that the wearable finger ring device enables getting a reliable estimate of Electrocardiogram (ECG)-Photoplethysmography (PPG, through which Pulse Transit Time (PTT) is measured. As the Pulse Transit Time (PTT) measurement is facilitated through the wearable finger ring device, the blood pressure measurement is achieved without the use of cuffs. Further, the miniature size of the wearable finger ring provides superior comfort along with minimal intrusion while maintaining quality of data. Other advantages of the present invention include monitoring of sleep, stress and activity using a combination of the one or more vitals by multi-sensor hardware in concordance with algorithms.

### BRIEF DESCRIPTION OF THE DRAWINGS

The foregoing and other features of embodiments will become more apparent from the following detailed description of embodiments when read in conjunction with the accompanying drawings. In the drawings, like reference numerals refer to like elements.

FIG. 1 illustrates the isometric view of the wearable finger ring device.

FIG. 2 illustrates the block diagram of the system including the wearable finger ring device and the receiving unit involved in the measurement of multiple vitals and pulse transit time.

FIG. 3 illustrates an example of the measurement of Pulse Transit Time (PTT) using a wave diagram of biometric signals.

### DETAILED DESCRIPTION OF THE INVENTION

Reference will now be made in detail to the description of the present subject matter, which are shown in figures. Various changes and modifications obvious to one skilled in the art to which the invention pertains are deemed to be within the spirit, scope and contemplation of the invention.

The terminology used in the description presented herein is not intended to be interpreted in any limited or restrictive way, simply because it is being utilized in conjunction with detailed description of certain specific embodiments of the invention. The terms “wearable finger ring device” and “device” may be interchangeably used. Further, the terms “Electrocardiogram (ECG) signal” and “biopotential” may be interchangeable. It is further understood that the terms “lead” and “electrode” may be interchangeable, when used herein, specify the device used for measurement of a parameter.

The present invention discloses a wearable finger ring device for measuring multiple vitals of the user. The device senses more than one vital of the user using multiple sensors including but not limited to Photoplethysmography (PPG) signal, Electrocardiogram (ECG) signal, Bioimpedance (BioZ) and temperature sensors, to extract the information including but not limited to heart rate, SpO₂, blood pressure, skin temperature, respiratory rate. The device measures the Photoplethysmography (PPG) signal, Electrocardiogram (ECG) signal through the finger of the user, facilitating measurement of the Pulse Transit Time (PTT) that is a marker for cuffless Blood Pressure (BP) measurement.

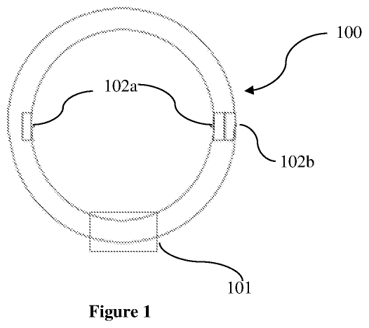

FIG. 1 illustrates an isometric view of the wearable finger ring device. The wearable finger ring device (100) discloses an optical sensing unit (101) located at the palm side of the finger and an electrode module (102) having at least one inner electrode (102a) on the same or opposite sides and an outer electrode (102b).

According to the preferred embodiment of the present invention, the optical sensing unit (101) comprises atleast one multi-wavelength light emitting diodes (LED) and a photodiode (PD) that are located at the palm side of the finger. The optical sensing unit (101) has at least one light emitting diode that faces the skin of the user, which illuminates the capillaries underneath to facilitate collection of the Photoplethysmography (PPG) signal. The photodiode (PD) of the optical sensing unit (101) faces the skin side of the finger that senses and collects the subtle changes in the reflected light in response to the blood flow.

The electrode module (102) comprises atleast one inner electrode (102a) and an outer electrode (102b), wherein the inner electrodes (102a) are located on the same or opposite sides of the inner surface, are in constant contact with the skin, that measures the Bioimpedance (BioZ). The outer electrode (102b) is located on external side in order to make contact with suitable measurement sites on the body, facilitating the measurement of Electrocardiogram (ECG) signal or Biopotential signal at multiple sites. The optical sensing unit (101) measures the Photoplethysmography (PPG) signal and the electrode module (102) measures the Electrocardiogramal through the electrodes, where the Electrocardiogram (ECG) signal and Photoplethysmography (PPG) signal is used in measuring the Pulse Transit Time (PTT) for cuffless blood pressure (BP) measurement.

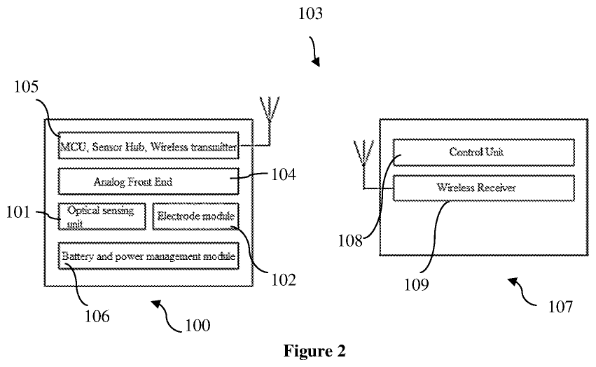

FIG. 2 illustrates a block diagram indicating the system including wearable finger ring device and the receiving unit involved in the measurement of multiple vitals and pulse transit time. The system (103) comprises a wearable finger ring device (100) and a receiving unit (107) where the wearable finger ring device (100) includes an optical sensing unit (101), electrode module (102) having more than one inner electrodes (102a) and an outer electrode (102b), a multi-sensor analog front end (104) coupled with a sensor hub and primary Microcontroller (MCU) and wireless transmitter (105) and battery and power management module (106).

The system (103) discloses the multi-sensor Analog Front End (AFE) (104) which processes and conditions all the collected signals to improve the signal quality. The multi-sensor analog front end (104) is coupled with a sensor hub and primary Microcontroller (MCU) and wireless transmitter (105) where the primary Microcontroller (MCU) and wireless transmitter (105) collects all the processed information from the multi-sensor Analog Front End (AFE) (104) and transmits the data to a receiving unit (107) for example: an electronic device through wireless network infrastructure such as Bluetooth, General Packet Radio Service (GPRS), Wi-Fi and other systems which fall within the purview of wireless network infrastructure. The battery and power management module (106) powers the device (100).

The system (103) discloses a receiving unit (107) having a control unit (108) and wireless receiver (109), where the receiving unit (107) is capable of transmitting and receiving data wirelessly to enable remote monitoring and wireless control of the device (100).

The wearable finger ring device (100) senses and measures the Photoplethysmography (PPG), Electrocardiogram (ECG), Bioimpedance (BioZ) and also senses the temperature of the user. The device (100) is capable of measuring multiple vitals of the user including heart rate, oxygen saturation (SpO₂), blood pressure, skin temperature, respiratory rate. The wearable finger ring device (100) is capable of monitoring sleep, stress and user activity using a combination of the above vitals with the help of algorithms.

According to the present invention, the method of estimation of the Pulse Transit Time (PTT) using the device (100) requires the user to raise the level of the device (100) to chest level in order to obtain the Electrocardiogram (ECG) signal accurately, which along with Photoplethysmography (PPG) signal is used in accurate estimation of the blood pressure. The device (100) facilitates multi-site Electrocardiogram (ECG) signal measurement in multi-lead configurations. The cuffless blood pressure estimation is achieved using Pulse Transit Time (PTT) and Pulse Wave Analysis (PWA).

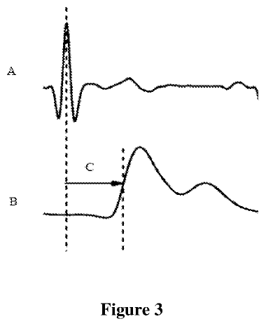

FIG. 3 illustrates an example of the measurement of pulse transit time (PTT) using a wave diagram. The Pulse transit time (PTT) is measured by using the measured values of Photoplethysmography (PPG) signal and Electrocardiogram (ECG) signal, where the Pulse transit time (PTT) is the time taken for the arterial pulse pressure wave to travel from the aortic valve to a peripheral site. A illustrates the graph of Electrocardiogram (ECG) and B illustrates the graph of Photoplethysmography (PPG), and C illustrates the Pulse transit time (PTT), that provides a basis for cuffless Blood Pressure (BP) measurement. Additionally, Pulse transit time (PTT) varies inversely with BP due to the physical properties of arteries and is estimated using the wearable finger ring device (100) without a cuff.

There are several advantages of the present invention including continuous measurement of multiple vitals including but not limited to Photoplethysmography (PPG), Electrocardiogram (ECG), body temperature and Bioimpedance (BioZ) through the wearable finger ring device (100), where the measurement is minimally intrusive. The device (100) has the ability to collect synchronous Photoplethysmography (PPG) signal and Electrocardiogram (ECG) signal through device (100) through which the Pulse Transit Time (PTT) estimation is extremely accurate. As the Pulse Transit Time (PTT) measurement is estimated through the device (100), the blood pressure measurement is achieved without the use of cuffs.

The wearable finger ring device (100) measures the Electrocardiogram (ECG) signal using small form factor dry electrodes and the inner electrodes (102a) singularly measure the Electrocardiogram (ECG) signal, Bioimpedance (BioZ) and body temperature of the user. Further, the miniature size of the wearable finger ring provides superior comfort while maintaining quality of data. The device (100) monitors the sleep, stress and activity using a combination of the one or more vitals collected by the device (100).

The applications of the wearable finger ring device (100) include estimation of Pulse Transit Time (PTT) which further leads to cuffless blood pressure (BP) measurement, the measurement and tracking of multiple biomarkers, measurement of multiple vitals including but not limited to body temperature and Bioimpedance (BioZ).

We claim:

1. A wearable system for measurement and tracking of multiple biomarkers, the system (103) comprising:
a. a wearable finger ring device (100) comprising:
i. an optical sensing unit (101) for facilitating measurement of the Photoplethysmography (PPG) signal, wherein the optical sensing unit (101) includes at least one multi-wavelength light emitting diodes (LED) and a photodiode (PD);
ii. an electrode module (102) comprising at least one inner electrode (102a) and an outer electrode (102b) for facilitating the measurement of Electrocardiogramals, wherein the inner electrodes (102a) singularly measure the Electrocardiogram (ECG) signal, Bioimpedance (BioZ) and body temperature of the user;
the device (100), wherein the optical sensing unit (101) and the electrode module (102) facilitate synchronous measurement of Photoplethysmography (PPG) signal and Electrocardiogram (ECG) signal at multiple sites, enabling the estimation of the Pulse Transit Time (PTT) and Pulse Wave Analysis (PWA) for the measurement and tracking of Blood Pressure (BP) in a cuffless manner.

2. The system (103) as claimed in claim 1, wherein the optical sensing unit (101) comprises:
the multi-wavelength light emitting diodes (LED) facing the skin of the user's finger, that illuminates the capillaries underneath for facilitating measurement of Photoplethysmography (PPG) signal;
a photodiode (PD) facing the skin of the user's finger, that senses subtle changes in the reflected light in response to the blood flow.

3. The system (103) as claimed in claim 1 wherein, the electrode module (102) includes at least one inner electrode (102a) and an outer electrode (102b) wherein:
the inner electrode (102a) is located on the inner surface of the wearable finger ring device (100) measures the bioimpedance (BioZ); and
the outer electrode (102b) is located on external side of the wearable finger ring device (100) facilitates multi-site Electrocardiogram (ECG) signal measurement in multi-lead configurations.

4. The system (103) as claimed in claim 1, wherein the wearable finger ring device (100) includes a multi-sensor Analog Front End (AFE) (104) coupled with a sensor hub, a primary microcontroller (MCU) and a wireless transmitter (105), where the multi-sensor AFE (104) processes and conditions all the collected signals.

5. The system (103) as claimed in claim 1, wherein the primary Microcontroller (MCU) and the wireless transmitter (105) collects the processed information from the multi-sensor AFE (104) and transmits the data through a wireless network infrastructure for remotely monitoring the wearable finger ring device (100).

6. The system (103) as claimed in claim 1, wherein a battery and power management module (106) supplies power to the wearable finger ring device (100).

7. The system (103) as claimed in claim 1, wherein the wearable finger ring device (100) further comprises a receiving unit (107) including a control unit (108) and a wireless receiver (109), wherein the receiving unit (107) receives data wirelessly through the wireless receiver (109) to enable remote monitoring and wireless control of the wearable finger ring device (100).

8. The system (103) as claimed in claim 1, wherein the wearable finger ring device (100) collects synchronous Photoplethysmography (PPG) signal and Electrocardiogram (ECG) signal used in the estimation of Pulse Transit Time (PTT) which in combination with Pulse Wave Analysis (PWA) of the PPG signal morphology enables cuffless Blood Pressure (BP) measurement.

9. The system (103) as claimed in claim 1 wherein, the data transmission from wearable finger ring device (100) to the receiver unit (107) is achieved through a wireless network infrastructure.
